(19) United States
(12) Patent Application Publication
(10) Pub. No.: US 2022/0315880 A1
(43) Pub. Date: Oct. 6, 2022

(54) BIOREACTORS AND METHODS OF USE

(71) Applicant: New Environmental Engineering, Inc., San Pedro, CA (US)

(72) Inventor: John Stewart Lang, Los Angeles, CA (US)

(21) Appl. No.: 17/701,141

(22) Filed: Mar. 22, 2022

Related U.S. Application Data
(60) Provisional application No. 63/168,577, filed on Mar. 31, 2021.

(51) Int. Cl. C12M 1/26; C12M 1/06; C12M 1/00; C12M 1/36

(52) U.S. Cl. CPC C12M 33/10; C12M 27/02; C12M 29/00; C12M 41/48

(57) ABSTRACT

The inventions described herein concern improved bioreactors and methods of aerating fluids.

### CROSS-REFERENCE TO RELATED APPLICATIONS

This application claims priority to, and incorporates by reference as if fully set forth herein, U.S. Provisional Application Ser. No. 63/168,577, which was filed on Mar. 31, 2021.

### FIELD OF THE INVENTION

The inventions described herein relate to improved bioreactors and methods of use to more efficiently grow microorganisms.

### SUMMARY OF THE INVENTION

Bioreactors are used to grow aerobic organisms used in products such as medicines, vaccines, cosmetics, and many others.

The inventions described herein may be used to increase oxygen levels in bioreactors with the intent of accelerating the growth rate of the microorganisms in said reactors.

In prior bioreactor designs, the sole option that the devices operators could use to increase the efficiency of oxygen transfer was to increase the rotational speed of the bioreactor's agitators. This control function is limited by the shear that the agitators create as they rotate. At a certain point, the shear forces become so great that it ruptures the cells growing in the bioreactor.

The current inventions teach the use of a centrifuge to enhance sedimentation of solid particles suspended in the fluid medium contained within a bioreactor. The supernatant from the centrifugal separator then flows through a venturi.

Bernoulli demonstrated the eponymous principal: the faster a fluid flows, the lower the pressure within the fluid becomes. This low pressure can be used to pull a gas (air or oxygen) into the venturi and mix the gas with the fluid flowing through the venturi. High shear forces within the venturi break the incoming gas into small bubbles.

In U.S. Pat. No. 10,246,359, there is described a derivation of this relationship:

Rt∝1/(Bd)²

Where Rt is the rate of gas transport from the interior of a submerged bubble to its surface and Bd is the diameter of the bubble. This inverse square proportionality demonstrates that decreasing the size of submerged bubbles is a very effective means of increasing gas transfer to the liquid phase.

After a gas molecule reaches the submerged bubble's surface it must dissolve into the liquid phase to complete the gas-liquid transport process. Bioreactor designers use a performance metric, kLa, to guide process and bioreactor design. In this metric, a is the aggregate submerged bubble surface area, and kL is the liquid side gas mass transfer coefficient.

Unpacking kL will illustrate how the inventions described herein are an improvement of current bioreactor designs. Several parameters are incorporated in the current understanding of kL. Some of these are: agitator geometry, agitator power input, agitation tip speed, and gas sparger design. All these parameters are eliminated in this novel invention by using a venturi to sparge gas into a bioreactor. With a venturi, the gas transfer rate into the bioreactor process depends on the gas concentration gradient across the gas-liquid interface. The primary parameters that influence this gradient are:
The bulk liquid concentration of the dissolved gas, Bc;
   the solubility of the gas at Bc, Sc; and
the flow rate at which liquid is moved away from the interface, Vl.
   Bc and Sc are operational parameters set by the process within the bioreactor. Vl is a function of the flow velocity within the venturi. Increasing the venturi flow velocity makes Vl larger and produces smaller gas bubbles.

With these insights, it is therefore possible to write a new definition of kLa that pertains to the operation of the novel bioreactors described in this invention:

kLa=F/(Bd)²

Where F is a function of Bc, Sc, and Vl.

The inventions described herein avoid the problems of the prior art by, among other things, separating the cells from the fluid before the fluid is aerated. In one embodiment, a bioreactor comprises a mixing tank containing cells to be cultured in a media. Paddle agitators are optionally attached to suitable crossbars and shafting is provided within the mixing tank to rotate the agitator assembly to keep the cells in suspension. A pump is coupled to a mixing tank to withdraw fluid and deliver it to an extractor that separates the cells from media by centrifugation. The cells collected at the periphery of this extractor, due to centrifugation, are fed back to the mixing tank, whereas the supernatant fluid left is pumped into a venturi that facilitates aeration of the supernatant fluid. The aerated supernatant fluid then flows back into the mixing tank. Additionally, in the proposed bioreactor the entrance point of the aerated fluid to the mixing tank may be such that the solids are kept in suspension in the mixing tank, thereby obviating the need for prior art agitators. The connections between the mixing tank, extractor and venturi are made by using suitable pumps, valves and pipes.

The most common bioreactor design is a continuously stirred tank reactor (CSTR). Both batch reactors and continuous flow-through reactors are in use. In a batch process, a bioreactor is partially filled with media. The cells are then inoculated and grow until the nutrients are consumed, and then harvested. A fed-batch process is similar to a batch process, except fresh nutrients are introduced to the bioreactor as required, which prevents the depletion of nutrients and provides for additional cell growth. Another process, known as perfusion, is used to remove unwanted compounds, allowing the process to last longer than fed-batch. Diaphragm or peristaltic pumps may be used in conjunction flow filters. Because of this waste flow of media out of the bioreactor there is a constant fresh feed of media into the bioreactor to maintain volume. However, this process is not continuous, as the bioreactor does not reach steady state as the cells grow and will reach a limit in cell density. A continuous process is when the bioreactor substantially reaches a steady state, where there is a continuous stream of feed into the bioreactor and a continuous outflow of product out of the bioreactor. The goal is to manufacture the product at a constant rate with little or no downtime between batches.

A batch reactor is shown in FIG. 1 for the purpose of illustration. The inventions disclosed herein pertain to batch, perfusion and continuous flow-through reactors.

### BRIEF DESCRIPTION OF THE DRAWINGS

FIG. 1 is a diagram view of a conventional bioreactor according to the prior art.

FIG. 2 is a diagram view of a first embodiment of a bioreactor according to embodiments of the inventions described herein.

FIG. 3 is a diagram view of a second embodiment of a bioreactor according to embodiments of the inventions described herein.

FIG. 4 is a flow chart illustrating the steps of a method for using bioreactors according to embodiments of the inventions described herein.

### DETAILED DESCRIPTION

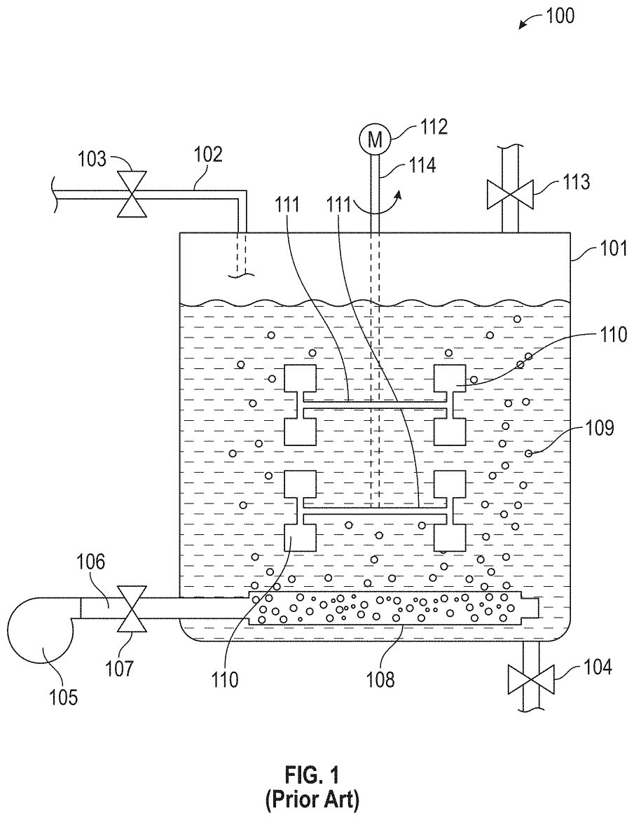

FIG. 1 illustrates a bioreactor 100 according to the prior art. Bioreactor mixing tank 101 is a vessel. An operator fills bioreactor mixing tank 101 by way of tank inlet 102, which is fitted with tank inlet valve 103. Tank inlet valve 103 may be selectively opened and closed by the operator. Similarly, the operator may drain bioreactor mixing tank 101 by selectively opening or closing tank drain valve 104. Blower 105 draws air in from a purified source into an air pipe 106 for the purpose of aerating bioreactor mixing tank 101's contents. The flow of this air is controlled by air manifold valve 107. Air from the blower flows through air pipe 106 and into a sparger 108 disposed within bioreactor mixing tank 101. Sparger 108 is most commonly a tube with holes drilled through it at intervals that allow air to escape, typically in the form of air bubbles as illustrated by bubble plume 109. Paddle agitators 110, also disposed within bioreactor mixing tank 101, are attached to suitable cross bars 111 and shaft 114 that connect them to a motor 112, that turn the paddle agitators 110 to stir the contents of bioreactor mixing tank 101 to increase the transfer of oxygen into bioreactor mixing tank 101's contents by agitating the rising air bubbles. The flow of air out of bioreactor mixing tank 101 is selectively controlled by air valve 113.

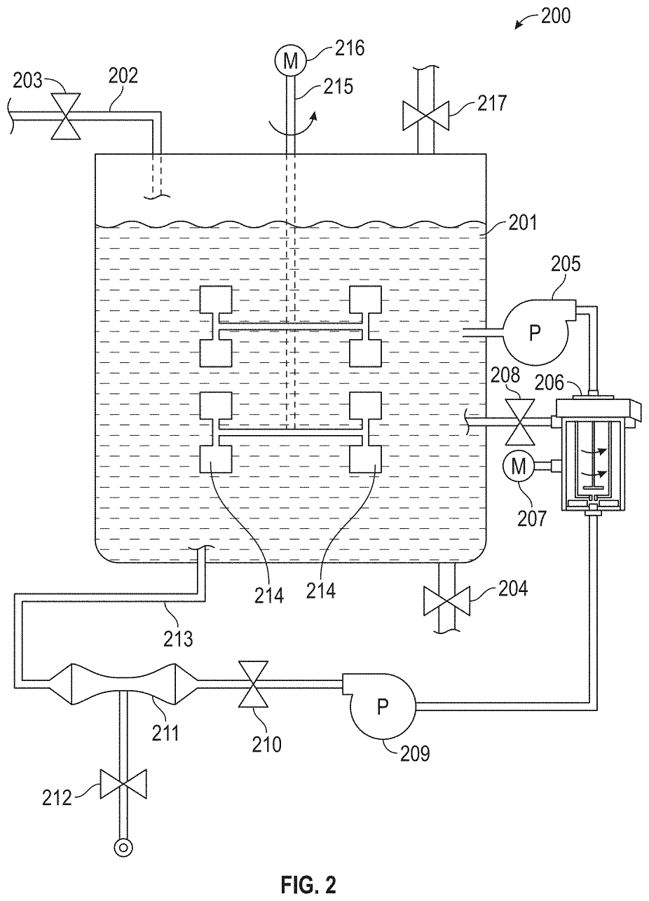

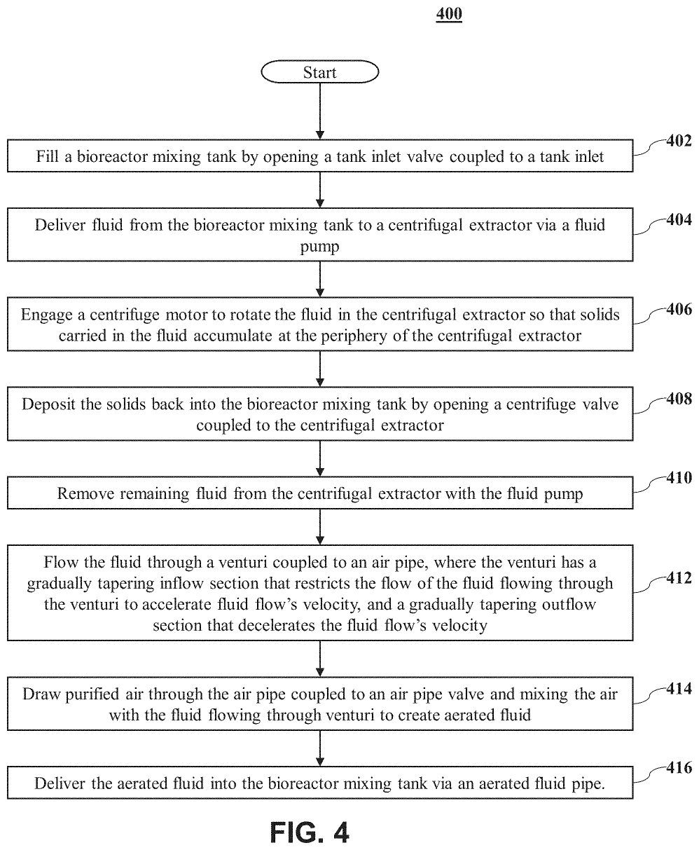

Bioreactor 200 as described herein solves the limitations of the prior art as described above. In one embodiment illustrated in FIG. 2, bioreactor mixing tank 201 is a vessel. A method for aerating a fluid 400 using bioreactor mixing tank 201 is detailed in the method steps set forth in FIG. 4. In step 402, an operator fills bioreactor mixing tank 201 by way of tank inlet 202, which is fitted with tank inlet valve 203. Tank inlet valve 203 may be selectively opened and closed by the operator to permit fluid to fill the tank. Similarly, the operator may drain bioreactor mixing tank 201 by selectively opening or closing tank drain valve 204. In one embodiment, bioreactor 200 may be operated using a continuous flow process, where the fluid entering bioreactor mixing tank 201 by way of tank inlet 202 creates an inlet stream, and opening tank drain valve 204 creates an outlet stream. When the inlet stream and outlet streams flow simultaneously at substantially the same rate, bioreactor 200 reaches a steady state of continuous flow. Depending on the application, this continuous flow may have several advantages, including no downtime between batches, no cleaning tasks, and no batch-to-batch variability.

In one embodiment, shown in step 404, pump 205 pulls a stream of fluid from bioreactor mixing tank 201 and delivers this fluid to a centrifugal extractor 206. In step 406, a centrifuge motor 207 is engaged that causes the contents of the centrifugal extractor 206 to rotate. Centrifugal force causes the solids carried in the fluid to accumulate at the periphery of the centrifugal extractor 206. This process is an enhanced form of sedimentation. It is inherently low shear and kills the live cells at a much lower rate than traditional bioreactor aeration methods. In step 408, the solids are selectively deposited back into bioreactor mixing tank 201 by controlling the opening and closing of centrifuge valve 208.

In step 410, the remaining fluid (without solids) in centrifugal extractor 206 may be removed from the centrifugal extractor 206 by fluid pump 209 and in step 412, delivered to venturi valve 210, which controls the flow of this fluid into a venturi 211 disposed in the bioreactor mixing tank 201. Venturi 211 is connected to an air pipe. Venturi 211 creates a vacuum that draws purified air through the air pipe, mixing the air with the fluid traveling through venturi 211, and aerating the fluid in step 414. Air pipe valve 212 controls the flow of purified air into venturi 211. The venturi 211 creates a low pressure zone that draws in purified air and mixes this air with the fluid flowing through the venturi 211. The high shear forces in the venturi 211 break the air in to small bubbles, which are highly efficient in aerating the fluid. The venturi 211 can be similar to the one described in U.S. Pat. No. 10,604,429, or it can be any type of venturi 211 that has a gradually tapering inflow section that restricts the flow area of the fluid flowing through the venturi 211. This restriction accelerates the fluids flow velocity. The fluids flow velocity is then decelerated by a gradually tapering outflow section that increases the fluid flow area.

In step 416, aerated fluid then delivered back into bioreactor mixing tank 201 via aerated fluid pipe 213. Agitators 214, when optionally coupled to a shaft 215 and disposed within bioreactor mixing tank 201, are not needed to assist oxygenation of the contents of tank. Rather, if included, they may be selectively moved by motor 216 at a speed sufficient to keep the solids in bioreactor mixing tank 201 in suspension, but low enough to not damage the cells. The flow of air out of bioreactor mixing tank 201 is controlled by air valve 217. In this embodiment, the aeration system is designed so that the fluid flow and the air flow can be controlled separately and independently. These controls can be operated manually. Alternatively, suitable sensors and electronic controllers can be used to operate the air and fluid control valves, including through automatic programming.

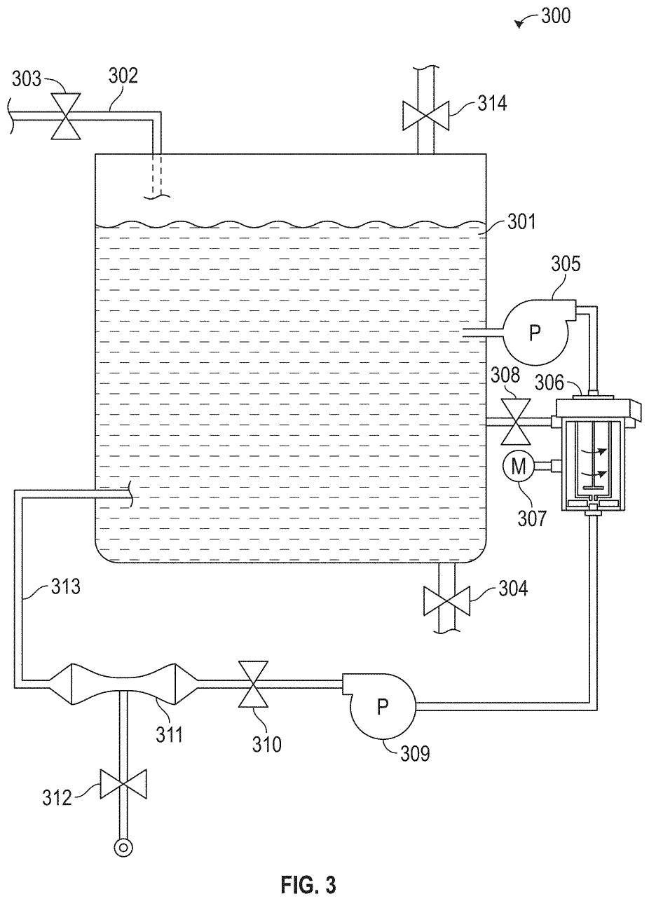

In yet another embodiment illustrated in FIG. 3, bioreactor 300 comprises a bioreactor mixing tank 301. Following the method steps detailed in FIG. 4, an operator fills bioreactor mixing tank 301 by way of tank inlet 302, which is fitted with tank inlet valve 303. Tank inlet valve 303 may be selectively opened and closed by the operator. Similarly, the operator may drain bioreactor mixing tank 301 by selectively opening or closing tank drain valve 304. Pump 305 pulls a stream of fluid from bioreactor mixing tank 301 and sends this fluid to a centrifugal extractor 306. Centrifuge motor 307 causes the contents of the centrifugal extractor 306 to rotate. Centrifugal force causes the solids carried in the fluid to accumulate at the periphery of the centrifugal extractor 306. This process is an enhanced form of sedimentation. The solids are selectively deposited back into bioreactor mixing tank 301 as controlled by centrifuge valve 308.

The remaining fluid (without solids) may be removed from the centrifugal extractor 306 by fluid pump 309. Venturi valve 310 controls the flow of this fluid into a venturi 311, which is connected to an air pipe. Venturi 311 creates a vacuum that draws purified air through the air pipe, mixing the air with the fluid traveling through venturi 311, and aerating the fluid. Air pipe valve 312 controls the flow of purified air into venturi 311. The flow of air out of bioreactor mixing tank 301 is controlled by air valve 314. In this embodiment, though, aerated fluid then flows back into bioreactor mixing tank 301 via aerated fluid pipe 313, which has a different entrance point on bioreactor mixing tank 301 than the one illustrated in FIG. 2. The effect of this change is to create a fluid circulation pattern in bioreactor mixing tank 301 (through rolling or otherwise) that serves to keep the solids in the tank in suspension. The entrance point of the aerated fluid shown in FIG. 3 is for illustrative purposes only and may be suitably selected by one of ordinary skill in the art to achieve this objective. This embodiment eliminates the need for agitators to be disposed within bioreactor mixing tank 301 to keep the solids in bioreactor mixing tank 301 in suspension.

While the invention has been described with reference to particular embodiments, it will be understood by those skilled in the art that various changes may be made and equivalents may be substituted for elements thereof without departing from the scope of the invention. In addition, many modifications may be made to adapt a particular situation or material to the teachings of the invention without departing from the scope of the invention.

Therefore, it is intended that the invention not be limited to the particular embodiments disclosed as the best mode contemplated for carrying out this invention, but that the invention will include all embodiments falling within the scope and spirit of the appended claims.

I claim:

1. A bioreactor comprising:
a bioreactor mixing tank having a tank inlet fitted with a tank inlet valve and a tank drain valve;
a first fluid pump configured to transfer fluid from bioreactor mixing tank to a centrifugal extractor, wherein the centrifugal extractor is coupled to a centrifuge motor and a centrifuge valve;
a second fluid pump configured to transfer a supernatant fluid from the centrifugal extractor to a venturi;
wherein the venturi is coupled to: (i) a venturi valve that controls the supernatant fluid's flow into the venturi, and (ii) an air pipe configured to deliver air to the venturi to aerate the supernatant fluid as it flows through the venturi to create aerated fluid; and
a fluid pipe for delivering aerated fluid into the bioreactor mixing tank.

2. The bioreactor of claim 1, further comprising one or more agitators disposed within the bioreactor mixing tank.

3. The bioreactor of claim 1, further comprising an electronic control system and sensors configured to operate the first fluid pump, the second fluid pump, and the centrifuge motor.

4. The bioreactor of claim 3, wherein the electronic control system and sensors are configured to open and close the tank inlet valve, the tank drain valve, the centrifuge valve, and the venturi valve.

5. A method for aerating fluid in a bioreactor mixing tank comprising the steps of:
filling a bioreactor mixing tank with a fluid by opening a tank inlet valve coupled to a tank inlet;
transferring the fluid from the bioreactor mixing tank to a centrifugal extractor coupled to a centrifuge motor and a centrifuge valve, via a fluid pump;
engaging the centrifuge motor to rotate the fluid in the centrifugal extractor to separate solids carried in the fluid and accumulate the solids at the centrifugal extractor's periphery;
opening the centrifuge valve coupled to the centrifugal extractor to deposit the solids into the bioreactor mixing tank;
activating the fluid pump to removing remaining fluid from the centrifugal extractor and deliver remaining fluid to a venturi coupled to an air pipe having an air pipe valve, wherein the venturi has a gradually tapering inflow section that restricts flow of the remaining fluid flowing through the venturi to accelerate the remaining fluid's flow velocity, and a gradually tapering outflow section that decelerates the remaining fluid's flow velocity;
drawing air through the air pipe via the air pipe valve, and mixing the air with the remaining fluid flowing through venturi to create aerated fluid; and
delivering the aerated fluid to the bioreactor mixing tank via an aerated fluid pipe.

6. The method of claim 5 further comprising the steps of:
opening a tank drain valve; and
draining the bioreactor mixing tank.

7. The method of claim 5 further comprising the steps of:
activating a motor coupled to an agitator via a shaft, wherein the agitator is disposed within the bioreactor mixing tank; and
rotating the agitator disposed within the bioreactor mixing tank.

8. The method of claim 7, further comprising the steps of:
rotating the agitator at a low speed sufficient to maintain the solids in the fluid in the bioreactor mixing tank in suspension.

9. The method of claim 6, further comprising the steps of:
operating the air pipe valve, the centrifuge valve, the tank inlet valve, and the tank drain valve using electronic controllers.

10. The method of claim 6, further comprising the steps of:
Operating the air pipe valve, the centrifuge valve, the tank inlet valve, and the tank drain valve manually.

11. The method of claim 5, further comprising the steps of:
creating a fluid circulation pattern in the bioreactor mixing tank that serves to keep the solids in the bioreactor mixing tank in suspension.

12. A method for continuously-aerating fluid in a bioreactor mixing tank comprising the steps of:
adding fluid to a bioreactor mixing tank by opening a tank inlet valve coupled to a tank inlet to create an inlet stream;
transferring the fluid from the bioreactor mixing tank to a centrifugal extractor coupled to a centrifuge motor and a centrifuge valve, via a fluid pump;
engaging the centrifuge motor to rotate the fluid in the centrifugal extractor to separate solids carried in the fluid and accumulate the solids at the centrifugal extractor's periphery;
opening the centrifuge valve coupled to the centrifugal extractor to deposit the solids into the bioreactor mixing tank;
activating the fluid pump to removing remaining fluid from the centrifugal extractor and deliver remaining fluid to a venturi coupled to an air pipe having an air pipe valve, wherein the venturi has a gradually tapering inflow section that restricts flow of the remaining fluid flowing through the venturi to accelerate the remaining fluid flow's velocity, and a gradually tapering outflow section that decelerates the remaining fluid flow's velocity;
drawing air through the air pipe via the air pipe valve, and mixing the air with the remaining fluid flowing through venturi to create an aerated fluid;
delivering the aerated fluid to the bioreactor mixing tank via an aerated fluid pipe; and
partially draining the bioreactor mixing tank by opening a tank drain valve to create an outlet stream, wherein both the inlet stream and outlet stream flow simultaneously at substantially the same rate.

13. The method of claim 12, further comprising the steps of:
Operating the air pipe valve, the centrifuge valve, the tank inlet valve, and the tank drain valve using electronic controllers.

14. The method of claim 12, further comprising the steps of:
operating the air pipe valve, the centrifuge valve, the tank inlet valve, and the tank drain valve manually.
